(19) United States
(12) Patent Application Publication
(10) Pub. No.: US 2008/0195153 A1
(43) Pub. Date: Aug. 14, 2008

(54) DYNAMIC SPINAL DEFORMITY CORRECTION

(76) Inventor: Matthew Thompson, Corte Madera, CA (US)

Correspondence Address: DERGOSITS & NOAH LLP, Suite 1450, Four Embarcadero Center, San Francisco, CA, 94111, US

(21) Appl. No.: 12/027,604

(22) Filed: Feb. 7, 2008

Related U.S. Application Data
(60) Provisional application No. 60/888,831, filed on Feb. 8, 2007.

(51) Int. Cl. A61B 17/58

(52) U.S. Cl. 606/257; 606/246; 606/254; 606/277

(57) ABSTRACT

A system for treating spinal deformities. A plurality of retaining clamps are fixed to a plurality of vertebrae and at least one elastic or super elastic rod is caused to pass through openings in the plurality of retaining clamps, the at least one elastic or super elastic rod being slidable within each of the plurality of retaining clamps along the axis of the spine to enable the spine to retain full flexion and extension in both its coronal and saggital planes.

### RELATED APPLICATION DATA

This nonprovisional utility patent application claims priority to provisional patent application No. 60/888,831, filed on Feb. 8, 2007.

### TECHNICAL FIELD

The present invention involves the use of dynamic stabilization techniques employing elastic or super elastic members captured by pedicle screws or like retaining clamps to reduce spinal deformities, such as scoliosis, over time. The present invention can be employed without resorting to spinal fusion or other immobilization techniques.

### BACKGROUND OF THE INVENTION

Spinal deformities are quite common generally affecting more girls than boys and manifesting itself during the teen years when significant growth is experienced. Scoliosis, the most common form of deformity, generally combines horizontal torsion and flexion in the frontal plane and develops in three spatial dimensions. As noted, the disease generally begins with the growth phase as it is hypothesized that this is probably due to the rotation of one or two vertebral bodies.

Sufferers of scoliosis are generally treated initially with a rigid corset like orthopedic brace. If this treatment proves unsuccessful, surgery is oftentimes resorted to. This involves the use of implantable apparatus including one and oftentimes two rods mounted in either side of the spinal column. If two rods are employed, anchoring means are provided positioning the rods in spaced-apart parallel alignment. Hooks or screws are employed to anchor the rods along the selected portion of the spinal column. Once installed, the anchors are rigidly locked to the associated rods to prevent relative motion therebetween and the entire arrangement supplemented with bone graphs causing fusion of the vertebra in the area in which the scoliosis has manifested itself. When fusion is resorted to, longitudinal connecting members are employed to resist flexion, extension, torsion, distraction and compression to substantially immobilize the portion of the spine that is fused. The longitudinal connecting members are designed to provide substantially rigid support in all planes.

Although spinal fusion can oftentimes largely correct a spinal deformity, such procedure is not without serious drawbacks. Spinal fusion can result in complications as the patient advances into adult life. The surgery requiring the application of bone graphs and permanent fixation of supporting clamps to the transverse process is significantly invasive. In addition, although fusion may result in strengthening a portion of the spine, it is also been linked to more rapid degeneration and collapse of spinal motion segments that are adjacent to the portion of the spine being fused, reducing or eliminating the ability of such spinal joints to move in a more normal relation to one another. Also, fusion has oftentimes failed to provide pain relief.

As with all such devices, the present invention employs retaining clamps fixed to a plurality of vertebra. Such retaining clamps are oftentimes in the form of pedicle screws applied to individual vertebra along at least the deformed segment of the spine. While there is a good deal of prior art dealing with dynamic stabilization using elastic members captured by pedicle screws, none of these devices are capable of reducing deformities over time. Regardless of whether the connecting rod between pedicle fixation points is elastic, once the pedicle screws have been firmly attached to the vertebra, the distance between those points will not change. In a spine with a healthy shape, that does not pose a problem. However, in a deformed spine, this fixes the deformity in place. A spine with a deformity in the coronal plane has a convex side and a concave side. The distance between the pedicles on the concave side is less than the distance between pedicles on the convex side. Oftentimes, patients experience symptoms from nerves that are being pinched by the spinal anatomy on the concave side of the deformity. It is apparent that fixing the distance between pedicles on either side fixes the deformity in place. A proper non-fusion deformity reduction system must be able to apply corrective forces, allow normal spinal motion and maintain application of corrective forces once the deformity begins to reduce.

Others have suggested improvements to the orthoses described above. For example, published U.S. Application No. 2004/0143264 teaches a system in which gliding or sliding rods are placed proximate the spinal axis employing dedicated retaining clamps capturing standard rods. This published application seeks only to afford some constrained motion following standard spinal surgery.

U.S. Pat. No. 7,125,410 teaches the use of elastic members designed to “resist buckling” and transmit axial loads. The disclosed structure does not allow axial motion of the sliding of rods. Screws which are employed are not standard or available pedicle screws but, are modified with certain features to mate with the disclosed elastic members and connectors. The disclosed construction does not actively compensate for creep or tissue relaxation and does not adequately treat deformities as it is taught that at least some of the rods are locked thus fixing the deformity.

U.S. Published Application No. 2007/0093814 teaches the use of stabilizing rods again not attachable to conventional pedicle screws and which do not allow for axial motion or the sliding of the rods. While the disclosed device allows for some motion of the spine, it has a defined limit noting that the specific disclosed example suggests 7 degrees. The system is not adequate in treating deformities as locking one or two rods with screws fixes the deformity and does not allow for correction.

U.S. Pat. No. 6,989,011 teaches a construction that limits spinal motion noting further that the corrective rods are locked in place and are therefore not capable of reducing the deformity.

U.S. Published Application Nos. 2007/005524 and 2004/0215192 teach devices which do not allow for axial translation. In the '524 publication, an outer sleeve is disclosed which is locked in the pedicle or bone screws and will not allow for deformity reduction. The '192 publication again does not allow for axial translation noting that the rods are locked in place on their respective retaining clamps.

U.S. Published Application No. 2006/0229612 teaches a system that allows for axial motion or “springs,” but there is no disclosed mechanism to retain the “springs” in extreme spinal flexion. The device disclosed in this publication will generally stabilize a normal spine quite well but is not adapted to reduce spinal deformities. While the device could initially offer some reduction in deformity, the length of the springs are fixed. Once some reduction occurs, a longer “spring” would be required to span the distance between the pedicle screws on the concave side of the deformity and a shorter “spring” would be required on the convex side.

U.S. Published Application No. 2005/0182409 teaches a system that utilizes a modified pedicle screw and cannot be employed with a standard screw of the type used herein. There is no disclosure of axial motion in the system noting that fixed initial lengths of the rods do not allow for continued correction of the deformity. Applicant views this concept more as a surgical technique than instrumentation that corrects a deformity. The disclosed axial member or rod is only there to stabilize temporarily while the osteotomies heal and fusion can potentially occur during this period.

U.S. Pat. No. 6,616,669 teaches a tethering system that can offer some initial correction but, as with most other systems discussed above, is based on instrumentation of a fixed length. Further correction would require shortening of the tethering cables disclosed therein.

Others have recognized the benefits that potentially present themselves by providing systems to correct spinal deformities without fusion. For example, such a system is disclosed in U.S. Pat. No. 6,554,831 providing the basis for a commercial embodiment known as the “Orthobiom System.” This system was actually made the subject of a laboratory investigation repeated in an article entitled The Influence of Fixation Rigidity on Intervertebral Joints—An Experimental Comparison Between a Rigid and Flexible System, J. Korean Neurosurg Soc 37:364-369 (2005) where a number of pigs were deformed by scoliosis and treated by this system. It was noted, however, that despite the intent to avoid fusion, “spontaneous fusion” did occur. The present invention, in employing highly flexible rods fully translatable between vertebra provides for full flexion and extension of the spine in both the coronal and saggital planes thus eliminating the “spontaneous fusion” observed by the referenced publication.

It is thus an object of the present invention to provide an appliance to correct spinal deformities while eliminating or significantly reducing the drawbacks of the prior art.

It is a further object of the present invention to provide a dynamic stabilization system capable of correcting spinal deformities without spinal fusion while using pedicle screws and similar retaining clamps commonly employed by others.

These and further objects will be more readily apparent when considering the following disclosure and appended claims.

### SUMMARY OF THE INVENTION

The present invention is directed to a system for treating spinal deformities comprising a plurality of retaining clamps fixed to a plurality of vertebra and at least one elastic or super elastic rod caused to pass through openings in said plurality of retaining clamps. The at least one elastic or super elastic rod being slidable within each of the plurality of retaining clamps along the axis of the spine, the flexibility of said at least one elastic or super elastic rod and its movement in said plurality of retaining clamps being sufficient to enable the spine to retain full flexion and extension in both its coronal and sagittal planes.

### BRIEF DESCRIPTION OF THE FIGURES

FIG. 1 is a perspective view of the portion of the spine bearing an embodiment of the orthoses of the present invention.

FIG. 2 is a perspective view showing an embodiment of the orthoses of FIG. 1 installed in a pedicle screw.

FIG. 3A is a side view of a pedicle screw employing a rod adapter and correction rod as an embodiment of the present invention.

FIG. 3B is a top view of the embodiment of FIG. 3A.

FIG. 4A is a top view of a spine bearing another embodiment of the orthoses of the present invention.

FIG. 4B is a side view of a possible configuration of an extension adapter employed in the orthoses of FIG. 4A.

FIG. 5 is a side view showing the use of a distraction shim employed as a preferred embodiment of the present invention.

FIGS. 6A and 6B are alternative embodiments showing a side view of another form of orthoses employing an extension adapter.

### DETAILED DESCRIPTION OF THE INVENTION

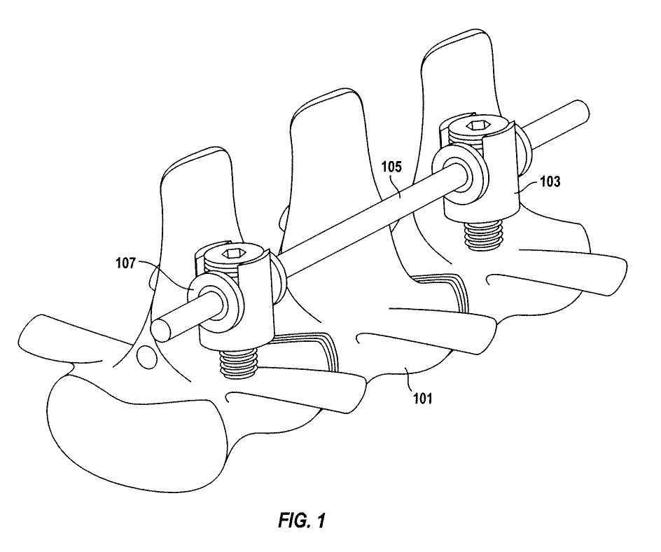

Turning first to FIG. 1, spine 101 is shown instrumented with pedicle screws 103. Installed in each pedicle screw is rod adapter 107. Elastic or super elastic corrective rods 105 are caused to pass through each rod adapter noting that the rod adapter freely allows axial translation of the corrective rods. The goal of this device is to transfer forces from the corrective rods to the vertebrae and spine through existing and available fixation devices while providing full flexion and extension in the spine's coronal and saggital planes, thus minimizing if not completely eliminating the possibility of spinal fusion. As will be more readily apparent in the discussion which follows, the present invention adapts to existing and available fixation systems using elastic or super elastic correction rods to slowly correct deformities without fusion while retaining full range of motion and full flexion and extension in the spine's coronal and saggital planes.

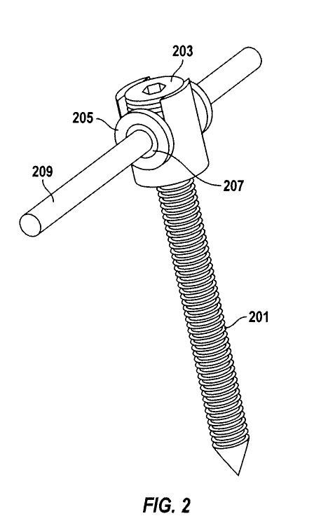

Rod adapter 107 can be seen in greater detail by making reference to FIG. 2. Specifically, pedicle screw 201 and set screw 203 are employed to capture rod adapter outer sleeve 205. In this embodiment, the outer sleeve has a retaining lip that prevents the adapter from axially slipping out of the pedicle screw. Captured inside the outer sleeve is inner bushing 207 that can be fabricated from known materials such as titanium alloy, polyethylene or various ceramics or other materials that will minimize friction and wear from motion of the elastic or super elastic correction rods that pass therein.

The rods used in practicing the present invention, in order to attain the goal of maintaining full flexion and extension in the coronal and saggital planes of the spine, are elastic or super elastic. Materials suitable for use in the manufacture of such rods include nitinol, shape memory alloys or polymers.

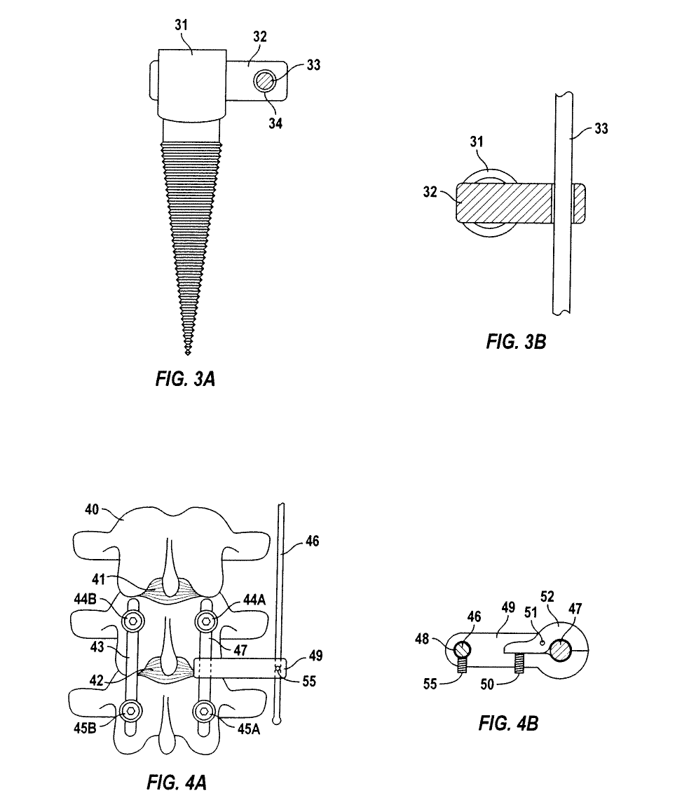

In further achieving the goals of the present invention, rods of varying sizes, such as 5.5 mm, 6.0 mm and ¼ inch diameter can be employed which may or may not be sized to adapt to standard pedicle screws of the type employed by the prior art. In order to accommodate certain patient pathologies or in using super elastic rods of diameters too large to pass through the head of a pedicle screw, the present invention can employ, as a preferred embodiment, offset adapters such as depicted in FIGS. 3A and 3B.

As an alternative to rod adapter 107 (FIG. 1), offset rod adapter 32 engages pedicle screw 31 so that correction rod 33 does not pass through the diameter of pedicle screw 31 but is instead offset a predetermined distance from the pedicle screw. In this way, opening 34 can be made of any size to facilitate the unencumbered, axial motion of rod 33 allowing the longitudinal axis of the spine to be corrected. Rod 33 in cross section together with opening 34 are shown in FIG. 3A as being circular allowing for not only the axial translation of rod 33 along the longitudinal axis of the spine but also allowing free rotation of the rod in the subject opening. As an embodiment of the present invention, the cross section of correction rod 33 can be made of a different shape, such as one which is oval with a mating oval-shaped opening 34 to substantially reduce if not eliminate rotational movement while still allowing axial translation.

Although there are various versions of rod adapter/pedicle screw combinations, reference is made to FIGS. 4A and 4B demonstrating the flexibility of the present invention.

In turning to FIG. 4A, spine 40 is shown in segment with vertebra 41 and 42 depicted. Pedicle screws 44A, 44B, 45A and 45B are of the standard variety shown installed with respect to vertebra 41 and 42. In this embodiment, rigid rods 43 and 47 bridge the pedicle screws as shown providing anchoring for offset rod adapter 49.

As further illustrative of the bridging of corrective rod 46 contained within opening 48 and rigid rod 47, reference is made to FIG. 4B where set screw 50 provides the adjustment to enable segment 52 to pivot at pin 51 thus enabling offset rod adapter 49 to selectively clamp upon rigid rod 47. Again, there are many and various mechanical options for achieving the net result of providing an offset adapter from spine 40, FIGS. 4A and 4B simply being illustrative of one of a myriad of such embodiments. Further, as a preferred and optional embodiment, a single set screw or locking nut 55 can be included to fix rod 46 at a single location to prevent the rod from migrating to the extreme ends of the spine.

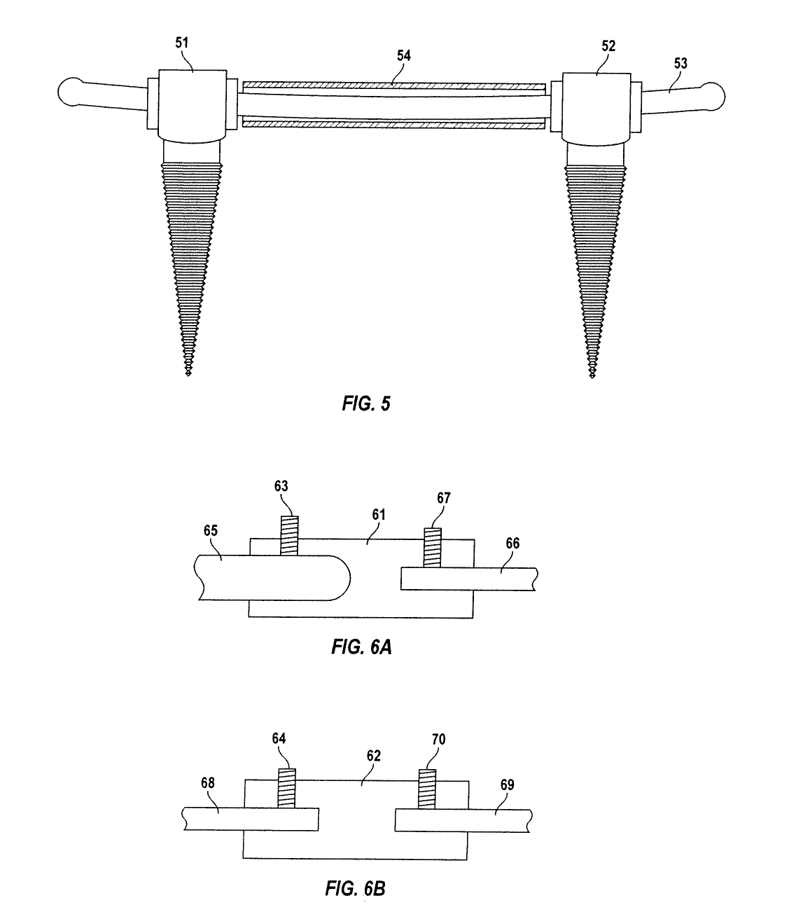

As yet a further embodiment of the present invention, reference is made to FIG. 5. As background, it has been recognized that it would be advantageous to provide for the ability to hold distraction across a segment of the spine being corrected while employing the present invention. Specifically, it is noted that on the concave side of the coronial plane of the deformity, distraction is key in opening the foramen to relieve pressure on existing nerve roots; this is because the distance between pedicles on the concave side is less than the distance between pedicles on convex side of the spine. Many times, patients have symptoms from nerves that are being pinched by spinal anatomy on the concave side of the deformity. The present invention can maintain axial motion of the correction rod while holding distraction by employing an embodiment such as that illustrated in FIG. 5.

FIG. 5 further depicts pedicle screws 51 and 52 intended to engage and be securely anchored to vertebra in a to-be corrected spine. Elastic or superelastic rod 53 is shown passing through pedicle screws 51 and 52 such as described in FIG. 1 above. However, in this embodiment, distraction shim 54 is employed on the diameter to facilitate free axial motion between correction rod 53 and distraction shim 54. It is noted that distraction shim 54, in the form of an elastic sleeve, is maintained between pedicle fixation point 51 and 52 such that the sleeve will act against the heads of these pedicle screws to hold the distraction shim while rod 53 is free to move axially. When rods are placed on either side of the spine, distraction shim 54 will be placed upon the correction rod 53 on the concave side of the spine for the reasons as discussed above.

In further recognition of the flexibility of the present invention, reference is made to FIGS. 6A and 6B. In this regard, it is known that most patients undergoing spinal surgeries for degenerative problems return in a few years with adjacent segment disease. Degeneration sometimes occurs in segments adjacent the surgically corrected spinal segments. For revisions of previous surgeries, both nonfusion and fusion, to adjacent levels of the device, connectors are provided to extend previous rods, both rigid fusion rods and elastic non-fusion rods. FIG. 6A pertains to the former while 6B pertains to the latter. In each instance, extension adapters 61/62 have openings to allow segments of rigid or corrective rods to be joined while providing a gap or spacing between ends of such rods acting to extend portions of the appropriate appliances. With regard to FIG. 6A, rigid rod 65 is shown being received by extension adapter 61 and maintained in place by set screw 63. Spaced apart from rigid rod 65 is corrective rod 66 again received by extension adapter 61 and held in place by set screw 67. Similarly, FIG. 6B shows extension adapter 62 receiving corrective rod 68 and corrective rod 69 held in place by set screw 64/70, respectively. Again, the space in between corrective rod 68 and 69 is determined by the length of extension adapter 62.

In summary, the improvements of spinal deformity correction employing the present invention are manifest. Such correction is made without spinal fusion and, in fact, through judicious use of elastic or super elastic corrective rods freely travelling through openings in and about adjacent pedicle screws, full flexion and extension in the coronal and saggital planes is maintained while avoiding spontaneous fusion of the type experienced in practicing the prior art.

What is claimed is:

1. A system for treating spinal deformities comprising a plurality of retaining clamps fixed to a plurality of vertebra and at least one elastic or super elastic rod caused to pass through openings in said plurality of retaining clamps, said at least one elastic or super elastic rod being slidable within each of said plurality of retaining clamps along the axis of the spine to enable the spine to retain full flexion and extension in both its coronal and saggital planes.

2. The system of claim 1 wherein said at least one elastic or super elastic rod corresponds to the shape of a corresponding part of a normal rachis of said spine.

3. The system of claim 1 wherein said at least one elastic or super elastic rod is immobilized in rotation in at least one of said plurality of retaining clamps.

4. The system of claim 1 wherein said plurality of retaining clamps are fixed to vertebra through the use of pedicle screws.

5. The system of claim 4 wherein an elastic rod adapter sleeve is maintained by said pedicle screw, the adapter sleeve retaining an inner bushing through which said at least one elastic or super elastic rod can slide.

6. The system of claim 1 wherein stoppers are placed proximate ends of said at least one elastic or super elastic rod to prevent said ends from passing through said retaining clamps.

7. The system of claim 1 further comprising at least one distraction sleeve between adjacent retaining clamps through which said at least one elastic or super elastic rod is slidable therethrough.

8. The system of claim 1 further comprising extension adapters secured to each of said retaining clamps, said extension adapters having an opening through which said at least one elastic or super elastic rod can slide.

9. The system of claim 1 further comprising rigid rods connecting adjacent retaining clamps and extension adapters secured to said rigid rods, each of said extension adapters having an opening through which said at least one elastic or super elastic rod can slide.

10. A system for treating spinal deformities comprising a plurality of retaining clamps fixed to a plurality of vertebra, extension adapters secured to each of said retaining clamps and at least one elastic or super elastic rod caused to pass through openings in said extension adapters, said at least one elastic or super elastic rod being slidable within each of said extension adapters along the axis of the spine to enable the spine to retain full flexion and extension in both its coronal and saggital planes.

11. A system for treating spinal deformities comprising a plurality of retaining clamps fixed to a plurality of vertebra and two elastic or super elastic rods caused to pass on opposite sides of said vertebra along the axis of the spine through openings in said plurality of retaining clamps, said two elastic or super elastic rods being slidable within each of said plurality of retaining clamps along said axis of the spine to enable the spine to retain full flexion and extension in both its coronal and saggital planes.

12. The system of claim 11 wherein said elastic or super elastic rods correspond to the shape of a corresponding part of a normal rachis of said spine.

13. The system of claim 11 wherein said elastic or super elastic rods are immobilized in rotation in at least one of said plurality of retaining clamps.

14. The system of claim 11 wherein said plurality of retaining clamps are fixed to vertebra through the use of pedicle screws.

15. The system of claim 11 wherein stoppers are placed proximate the ends of said elastic or super elastic rods to prevent said ends from passing through said retaining clamps.

16. The system of claim 11 wherein said spinal deformity is characterized as having at least one segment along said spine that is concave and wherein a distraction sleeve is placed between adjacent retaining clamps at said concave segment through which said elastic or super elastic rod is slidable therethrough.

17. The system of claim 11 further comprising extension adapters secured to each of said retaining clamps, said extension adapters having openings through which said elastic or super elastic rods can slide.

18. The system of claim 11 further comprising rigid rods connecting adjacent retaining clamps and extension adapters secured to said rigid rods, each of said extension adapters having openings through which said elastic or super elastic rods can slide.

19. A system for treating spinal deformities comprising a plurality of retaining clamps fixed to a plurality of vertebra, extension adapters secured to said retaining clamps, a pair of elastic or super elastic rods caused to pass on opposite sides of said vertebra along the axis with the spine through openings in said extension adapters, said two elastic or super elastic rods being slidable within each of said plurality of extension adapters along the axis of the spine to enable the spine to retain full flexion and extension in both its coronal and saggital planes.

20. A system for treating spinal deformities comprising a plurality of retaining clamps fixed to a plurality of vertebra and at least one elastic or super elastic rod caused to pass through openings in said plurality of retaining clamps, said at least one elastic or super elastic rod being suitable within all but one of said plurality of retaining clamps along the axis of the spine to enable the spine to retain full flexion and extension in both its coronal and saggital planes.

21. The system of claim 20 wherein said elastic or super elastic rod is fixed in a single of said retaining clamps.

22. The system of claim 20 wherein said elastic or super elastic rod is fixed in a single of said retaining clamps through use of set screw or locking nut.
